(19) United States
(12) Patent Application Publication
(10) Pub. No.: US 2026/0207807 A1
(43) Pub. Date: Jul. 23, 2026

(54) OZONE DISINFECTION DEVICE WITH AN OZONE RECOVERY MODULE

(71) Applicant: DCSTAR INC, New York, NY (US)

(72) Inventors: David LUO, New York, NY (US); Ligui HE, New York, NY (US)

(73) Assignee: DCSTAR INC, New York, NY (US)

(21) Appl. No.: 19/030,253

(22) Filed: Jan. 17, 2025

(51) Int. Cl. A61L 2/20; A61L 2/24; A61M 16/08; B01D 46/00; B01D 53/04

(52) U.S. Cl. CPC A61L 2/202; A61L 2/24; A61M 16/08; B01D 46/0043; B01D 53/0446; A61L 2103/15; A61L 2202/11; A61L 2202/13; A61L 2202/14; A61L 2202/15; A61M 2209/10; B01D 2253/306; B01D 2253/311; B01D 2257/106

(57) ABSTRACT

An ozone disinfection device with an ozone recovery module, the device being further configured to connect to a hose or respiratory device to disinfect the hose or the respiratory device. In this disclosure, the ozone disinfection device includes the ozone recovery module that includes a filter. A total surface area, a specific surface area, and a porosity of at least one filter material in the filter are defined to increase the filter's recovery efficiency. Furthermore, the filter may also be detachable, in cooperation with a housing structure, enabling a user to inspect or replace the filter independently and thus prolonging the overall service life of the ozone disinfection device. In some embodiments, the ozone disinfection device also includes at least one sensor and an alarm system to achieve intelligent control and prevent potential hazards.

### TECHNICAL FIELD

This disclosure relates to an ozone disinfection device configured to disinfect a respiratory device and its accessories. The device is configured to distribute ozone into a pipeline connectable to the respiratory device and/or its accessories, and to release ozone for cleaning and disinfection.

### BACKGROUND

Obstructive Sleep Apnea (OSA) is a common sleep-related breathing disorder. It manifests with various symptoms, such as breathing pauses during sleep, nighttime awakenings due to choking, daytime sleepiness, night sweats, attention deficits, and other issues. OSA can also lead to multiple complications, causing unpredictable harm to both the physical and psychological well-being of users. OSA may result in complications such as cardiovascular diseases, including hypertension (especially nighttime and morning hypertension), coronary artery disease, myocardial infarction, atrial fibrillation, heart failure, and nocturnal angina. It is also associated with endocrine and metabolic disorders, such as insulin resistance, type 2 diabetes, dyslipidemia, and metabolic syndrome. These metabolic issues can further exacerbate OSA symptoms, creating a vicious cycle. Neurological complications are also common, with OSA potentially leading to cognitive impairments, memory loss, attention deficits, and mood disorders such as anxiety and depression. Prolonged sleep deprivation and intermittent hypoxia may further increase the risk of stroke. Effective treatment of OSA can improve its primary symptoms and associated complications. Treatment options include lifestyle changes, continuous positive airway pressure (CPAP) therapy, and the use of oral appliances for orthodontic correction. In severe cases of OSA, surgical intervention may be necessary. Apart from surgical treatment, the aforementioned methods typically require long-term management and ongoing use of therapeutic devices. Therefore, these devices must be both effective and suitable for long-term use by users.

CPAP therapy is the most widely accepted and preferred treatment method for obstructive sleep apnea. The components required for CPAP therapy include a CPAP device (i.e., a ventilator), tubing, a mask, and a headgear. The mask typically consists of a support section and a sealing section and comes in various styles to meet different user needs and preferences. These styles include nasal pillows, nasal masks, full-face masks, and oronasal masks. The choice of mask should take into account factors such as sleeping position, facial structure, and personal preferences, as selecting a suitable mask can influence the effectiveness of CPAP therapy.

Ozone is a strong oxidizing agent capable of effectively eliminating bacteria, viruses, mold, and other microorganisms, achieving deep cleaning. It is particularly suitable for ventilatory devices that are challenging to clean thoroughly. After ozone is used for disinfection, it eventually decomposes into oxygen, leaving no harmful chemical residues. This makes ozone cleaning more environmentally friendly compared to methods using chemical disinfectants.

Adopting an appropriate disinfection method can prevent respiratory infections or complications caused by bacteria, mold, and viruses growing on device surfaces or inside tubing that may be carried into the user's airway through airflow. For individuals with allergies, the accumulation of dust, pollen, or other allergens inside uncleaned device tubing poses significant risks, potentially triggering respiratory allergies or asthma. Regular cleaning and disinfection can also reduce odors from the device, ensure fresher airflow, and improve the user experience, thereby enhancing compliance with treatment.

### SUMMARY

This disclosure addresses the aforementioned shortcomings by providing an ozone disinfection device that is user-friendly, cost-effective, and comfortable to wear.

In one embodiment, an ozone disinfection device with an ozone recovery module is provided. The ozone disinfection device includes an ozone generating module, the ozone recovery module, and a control system. The ozone disinfection device is configured to be connectable to a hose or a respiratory device to disinfect the hose or the respiratory device. The ozone generating module is configured to generate and output ozone gas. The ozone recovery module, located downstream of the ozone generating module, includes a suction device and a filter and is configured to draw in and filter the ozone gas. The control system is configured to control the operation of the components of the ozone disinfection device. The ozone generating module, the suction device, and the filter are configured to be in communication with one another, and the filter includes a filter material. The filtration efficiency of the ozone recovery module is positively correlated with the total surface area of the filter material.

In one embodiment, the control system further includes at least one sensor.

In one embodiment, the ozone recovery module further includes at least one exhaust port, the at least one exhaust port being configured to discharge gas.

In one embodiment, the at least one exhaust port is located downstream of the filter.

In one embodiment, during the operating period of the ozone disinfection device, the volume of gas drawn in by the suction device is greater than or equal to the volume of ozone gas generated by the ozone generating module during the same period.

In one embodiment, the filter is configured to be replaceable.

In another embodiment, an ozone disinfection device with an ozone recovery module is provided. The ozone disinfection device includes an ozone generating module, an ozone recovery module, and a control system. The ozone disinfection device is configured to be connectable to a hose or a respiratory device to disinfect the hose or the respiratory device. The ozone generating module is configured to generate and output ozone gas. The ozone recovery module, located downstream of the ozone generating module, includes a suction device and a filter and is configured to draw in and filter the ozone gas. The control system is configured to control the operation of the components of the ozone disinfection device. The filter includes a filter material, and the filter material has a specific surface area in a range of 5 m²/g to 500 m²/g.

In one embodiment, the ozone generating module, the suction device, and the filter are configured to be in communication with one another.

In one embodiment, the control system includes at least one sensor.

In one embodiment, the at least one sensor is configured to detect ozone concentration.

In one embodiment, the filter is located downstream of the ozone generating module.

In one embodiment, during the operating period of the ozone disinfection device, the suction device and the filter are configured to operate and cease operation simultaneously.

In yet another embodiment, an ozone disinfection device with an ozone recovery module is provided. The ozone disinfection device includes an ozone generating module, an ozone recovery module, and a control system. The ozone disinfection device is configured to be connectable to a hose or a respiratory device to disinfect the hose or the respiratory device. The ozone generating module is configured to generate and output ozone gas. The ozone recovery module, located downstream of the ozone generating module, includes a suction device and a filter and is configured to draw in and filter the ozone gas. The control system is configured to control the operation of the components of the ozone disinfection device. The filter is configured to have a porosity in a range of 10% to 95%.

In one embodiment, the ozone generating module, the suction device, and the filter are configured to be in communication with one another.

In one embodiment, the control system includes at least one sensor.

In one embodiment, the ozone generating module and the ozone recovery module are arranged in different compartments within the ozone disinfection device.

In one embodiment, the ozone generating module and the ozone recovery module are arranged in the same compartment within the ozone disinfection device.

In a further embodiment, an ozone disinfection device with an ozone recovery module is provided. The ozone disinfection device includes an ozone generating module, an ozone recovery module, and a control system. The ozone disinfection device is configured to be connectable to a hose or a respiratory device to disinfect the hose or the respiratory device. The ozone generating module is configured to generate and output ozone gas. The ozone recovery module, located downstream of the ozone generating module, includes a suction device and a filter and is configured to draw in and filter the ozone gas. The control system is configured to control the operation of the components of the ozone disinfection device. The ozone generating module, the suction device, and the filter are configured to be in communication with one another. The control system includes at least one sensor.

In one embodiment, the ozone recovery module further includes at least one exhaust port, with the at least one exhaust port configured to discharge gas.

In one embodiment, the filter includes a filter material, and the filter material has a filtration efficiency of at least 70%.

In one embodiment, the filter material has a porosity in a range of 50% to 85%.

In one embodiment, the at least one sensor includes one or more of an ozone concentration sensor, a temperature and humidity sensor, a pressure sensor, an oxygen sensor, a flow sensor, a volatile organic compound sensor, a particulate sensor, a current sensor, or a safety lock sensor.

Implementing the ozone disinfection device of the present disclosure has at least the following beneficial effects:
1. Compared to existing ozone disinfection devices on the market, the ozone disinfection device of the present disclosure employs more efficient filtration technology, delivering an enhanced user experience and achieving more thorough removal of ozone. As a result, the discharged gas is more environmentally friendly. Specifically, the filter of this disclosure uses optimized materials and structural designs, which may include multiple combined layers to realize different filtration functions (such as a physical filtration layer and a chemical adsorption layer). In addition, its structure is arranged to effectively improve ozone decomposition and filtration efficiency. Furthermore, the optimized filter design in the present disclosure reduces the need for frequent replacement of either the device or the filter, maximizing the operational effectiveness of each filtration layer. This lowers user costs and material waste, thereby decreasing maintenance expenses and making the device more eco-friendly. Through this design, the overall performance of the ozone disinfection device is improved while aligning with environmental protection and sustainable development requirements. Not only does it achieve a functional breakthrough, but it also contributes positively to environmental conservation, resource saving, and sustainability, making the device a more efficient, safer, and eco-friendly disinfection solution.
2. In the present disclosure, various parameters of the filter material are specified to achieve optimal filtration performance. First, the specific surface area of the filter material is controlled within a range of 5 m²/g to 500 m²/g. and preferably within a range of 50 m²/g to 200 m²/g. This not only achieves a better balance between the performance and cost of the ozone recovery module but also enhances the adaptability and application range of the filter material. A filter material with a higher specific surface area provides a larger surface for capturing and adsorbing ozone gas, thereby ensuring a higher level of cleanliness. Conversely, a filter material with a moderate specific surface area delivers effective filtration without creating excessive flow resistance, allowing air to pass smoothly through the ozone filter material. Through extensive experimental validation, preferably, the effective range for the specific surface area of the filter material is determined to be in a range of 5 m²/g to 500 m²/g. The optimization of the specific surface area achieves improved performance in terms of filtration efficiency, multifunctionality, airflow resistance, cost optimization, service life, and environmental sustainability. Furthermore, the porosity of the filter is specified to range from 10% to 95%, preferably from 50% to 85%. A higher porosity indicates more voids in the material, providing greater airflow space and reducing flow resistance. However, this may compromise the structural stability of the filter material, reducing its durability and potentially lowering filtration effectiveness. Conversely, a lower porosity is configured to more effectively adsorb or capture additional ozone gas. However, excessively low porosity may result in overly narrow filtration channels, restricting airflow and reducing the filtration efficiency of the ozone recovery module. If the porosity is too low, the filter material may also become clogged, decreasing its long-term reliability. Through extensive experimentation, it has been determined that the specified range of porosity achieves an optimal balance between filtration performance, reliability, and other critical factors. Additionally, it is established that, during the operating period of the ozone disinfection device, the volume of gas drawn in by the suction device must be greater than or equal to the volume of the ozone gas generated by the ozone generating module during the same period. This ensures the effective operation of the suction device during use, preventing the release of ozone gas at excessively high concentrations into the external environment, which could pose harm to both the environment and the user.
3. The present disclosure integrates sensors into the ozone disinfection device, enhancing its intelligence, automation, and safety. This optimization improves the disinfection effect while ensuring safety and environmental protection during use. The sensors include, but are not limited to, one or more of the following: an ozone concentration sensor, a temperature and humidity sensor, a pressure sensor, an oxygen sensor, a flow sensor, a volatile organic compound (VOC) sensor, a particulate sensor, a current sensor, or a safety lock sensor. The integration of these sensors not only enhances the multifunctionality of the device but also provides users with a more intelligent and efficient operational experience. Specifically, the ozone concentration sensor is configured to monitor the ozone concentration within the device in real time, ensuring that the ozone concentration during disinfection or recovery remains at appropriate levels. This prevents insufficient disinfection due to low ozone concentrations or harm to humans or the environment caused by excessively high concentrations. Furthermore, the ozone concentration sensor can detect whether the ozone disinfection device is leaking, thereby ensuring the effectiveness of the device's disinfection process. The temperature and humidity sensor ensures that the conditions for ozone disinfection are suitable and optimizes the disinfection environment, thereby improving the efficiency of ozone disinfection. The pressure sensor and flow sensor enable precise control of airflow distribution within the disinfection space, ensuring that ozone gas effectively reaches every part of the components being disinfected for thorough disinfection. The oxygen sensor detects the oxygen content in the recovered ozone disinfection space, helping to verify whether the ozone has been fully recovered, which benefits environmental protection and green design. Additionally, the oxygen sensor can monitor and adjust the efficiency of ozone generation, thereby optimizing the effectiveness of ozone disinfection. Other sensors, such as VOC sensors and particulate sensors, further enhance the device's ability to monitor its internal environment and make real-time adjustments to address potential contamination. The integration of current sensors and safety lock sensors further improves the safety of the device, preventing misuse and ozone gas leakage. The inclusion of one or more of these sensors enhances the intelligence and reliability of the device, providing users with a safer, more reliable, and more effective ozone disinfection solution.
4. The filter in the ozone disinfection device of the present disclosure is configured to be replaceable, offering multiple advantages such as facilitating the maintenance and management of the ozone disinfection device, as well as enhancing its disinfection efficiency and service life. First, the replaceable filter increases the variety of filters that can be used with the ozone disinfection device. Users can match filters with different internal filtering materials based on various usage scenarios and specific needs. For example, standard ozone filters can be used by general users in everyday environments. Conversely, users who frequently use ozone disinfection devices or disinfect heavily polluted devices may select filters with more efficient filtering materials. Additionally, users can choose filters with enhanced specific functions depending on the environment. For instance, in humid conditions, filters with enhanced drying functionality can be used. This approach reduces the impact of environmental factors on the filter in the ozone disinfection device, thereby improving the device's service life and disinfection efficiency to a certain extent. The ozone filter, as one of the most important functional components of the ozone disinfection device, offers additional benefits as technology advances. Users can upgrade the ozone disinfection device by replacing the filter with a more efficient or multifunctional one, providing advantages for both users and manufacturers. This approach not only reduces costs but also enhances the adaptability and versatility of the device. Furthermore, users can replace the filter independently based on their needs without requiring professional assistance, making the maintenance of the ozone disinfection device simpler and more convenient. In one approach, users can visually monitor the status of the filter and easily determine when replacement is needed, providing a more intuitive and user-friendly experience. Regular and straightforward replacement of the filter by the user prevents performance degradation caused by blockages or aging, effectively protecting and extending the service life of the entire ozone disinfection device. Finally, this method represents an environmentally friendly design for ozone disinfection devices. Instead of replacing the entire ozone disinfection device, only the smaller component—the ozone filter—needs to be replaced. This reduces the number of discarded devices and allows the ozone disinfection device to be reused multiple times with new filters. This approach aligns with modern green design principles and is more environmentally friendly.

### BRIEF DESCRIPTION OF THE DRAWINGS

FIG. 1 is a perspective schematic diagram of the ozone disinfection device in accordance with one embodiment of the present disclosure;

FIG. 2 is a schematic diagram of another form of the ozone disinfection device in accordance with one embodiment of the present disclosure;

FIG. 3 is a schematic diagram of the space accommodating various functional components of the ozone disinfection device in accordance with one embodiment of the present disclosure;

FIG. 4 is a schematic diagram of the functional components of the ozone disinfection device in accordance with one embodiment of the present disclosure;

FIG. 5 is a schematic diagram showing the adsorption of ozone gas by the filter material in the filter in accordance with one embodiment of the present disclosure;

FIGS. 6A and 6B are schematic diagrams showing the use of different materials as the filter material in the filter in the first embodiment of the present disclosure;

FIGS. 7A and 7B are schematic diagrams showing the varying adsorption capacities of filter materials with different specific surface areas for ozone gas in accordance with one embodiment of the present disclosure;

FIG. 8 is a schematic diagram showing the filter with cotton or mesh on both sides in accordance with one embodiment of the present disclosure;

FIGS. 9A and 9B are schematic diagrams showing filter materials with different porosities in the filter in accordance with one embodiment of the present disclosure;

FIG. 10 is a simplified diagram summarizing the components of the ozone disinfection device in accordance with one embodiment of the present disclosure;

FIG. 11 is a simplified diagram summarizing the components with a sensor inside the ozone disinfection device in accordance with one embodiment of the present disclosure;

FIG. 12 is a schematic diagram showing the replaceable filter in the ozone disinfection device in accordance with one embodiment of the present disclosure.

### DETAILED DESCRIPTION

To make the objectives, features, and advantages of this disclosure clearer and easier to understand, the specific embodiments of this disclosure are described in detail with reference to the accompanying drawings. Numerous specific details are provided below to facilitate a thorough understanding of the disclosure. However, the disclosure can be implemented in many different ways other than those described here, and those skilled in the art may make similar modifications without departing from the essence of the disclosure. Accordingly, this disclosure is not limited by the specific embodiments provided below.

Compared to existing ozone disinfection devices on the market, the present disclosure achieves more thorough and efficient ozone filtration by carefully designing and rigorously testing the ozone recovery module. As a result, it enables a safer and more environmentally friendly method of ozone emission, fully complying with strict environmental standards. Consequently, this disclosure provides a more eco-friendly ozone disinfection solution.

Additionally, in one embodiment, the disclosure integrates a sensor that works in tandem with a high-efficiency filter. On one hand, the sensor provides precise data at each stage of operation, thereby enhancing the device's effectiveness and reliability. On the other hand, this intelligent design extends the device's overall lifespan and ensures a more streamlined and smarter user experience. By combining intelligence, efficiency, and environmental friendliness, the ozone disinfection device described herein offers users an advanced, comfortable, and user-friendly disinfection experience.

Detailed embodiments are presented below to elucidate the configurations of the ozone disinfection device 1.

### Embodiment 1

The present disclosure provides an ozone disinfection device 1 that is convenient for users, enhances user convenience, and is comfortable to wear.

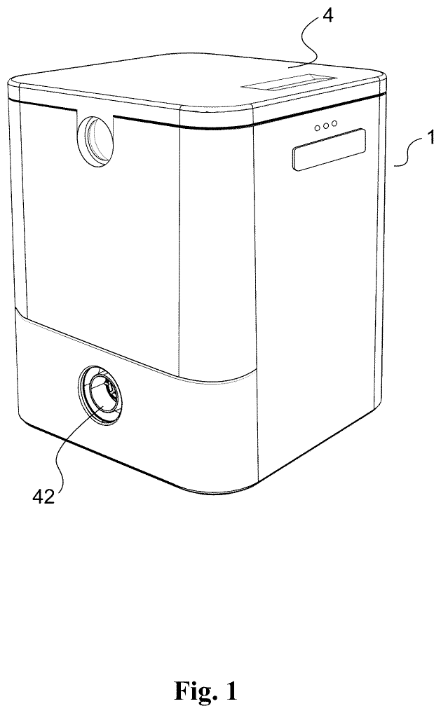

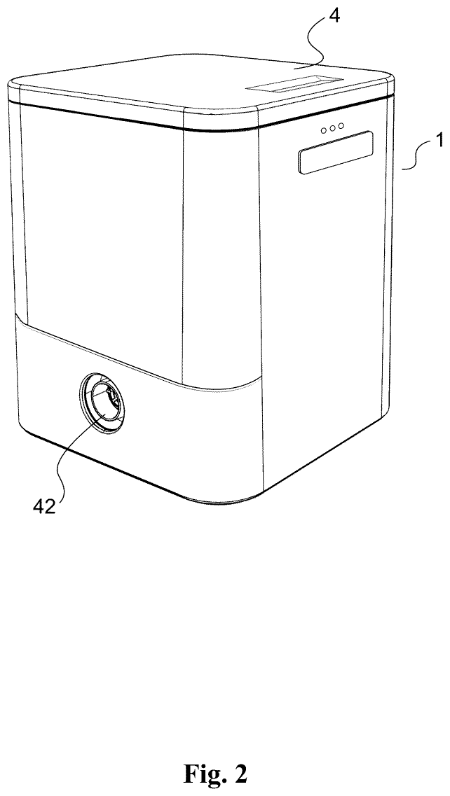

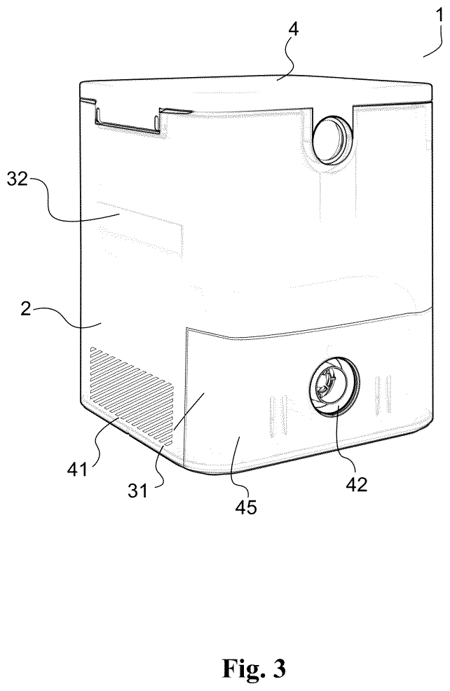

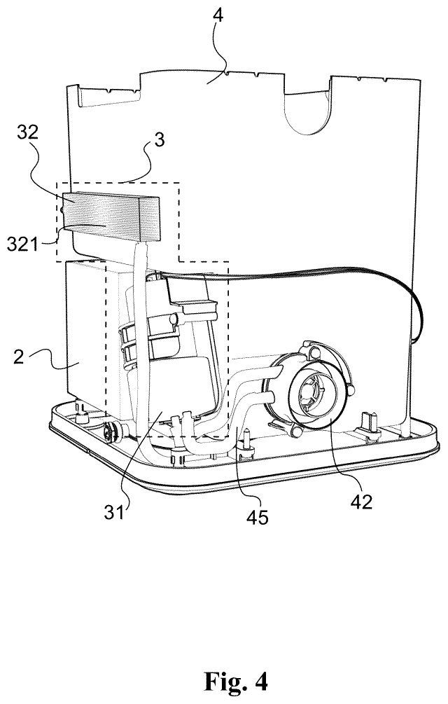

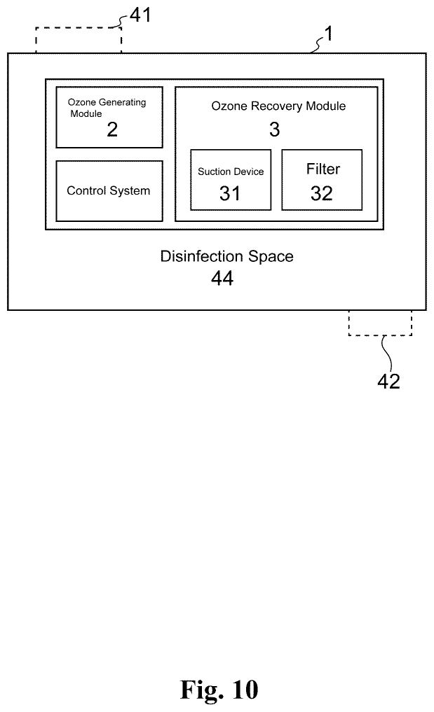

Referring to FIGS. 1 to 10, the ozone disinfection device 1 of the present disclosure includes: an ozone generating module 2 configured to generate and output ozone gas; an ozone recovery module 3, located downstream of the ozone generating module 2, the ozone recovery module 3 including a suction device 31 and a filter 32, and configured to draw in and filter ozone gas; a control system configured to control the operation of various components of the ozone disinfection device 1.

Specifically, the ozone generating module 2, the ozone recovery module 3, and the control system are functional components of the ozone disinfection device 1. These functional components are configured to be located within a housing 4. The housing 4 is configured to form the exterior of the ozone disinfection device 1 and is also configured to protect internal components. Additionally, the housing 4 provides stable structural support, ensuring stability of the device during use and transport. The housing 4 is formed by multiple walls. In some embodiments, the walls of the housing 4 include an upper cover and a lower cover, where the upper cover is configured to be openable. The opening mechanism may be a flip type, a sliding type, or any other form. In other embodiments, the housing 4 may be shaped like a drawer or have another form. In certain embodiments, the housing 4 also at least forms a disinfection space 44. The portion of the housing 4 defining the disinfection space 44 and the portion that accommodates the internal functional components may be the same housing 4. The disinfection space 44 is configured to hold items or accessories requiring disinfection, and to release ozone gas within this sealed space for disinfection. Generally, the disinfection space 44 and the functional components inside the housing 4 are positioned in two separate compartments. However, in specific cases, the disinfection space 44 and the compartment containing the functional components may be in communication with each other. In other embodiments, the disinfection space 44 may be formed by another housing 4 that is separate from the housing 4 surrounding the functional components. In this scenario, the housing 4 that defines the disinfection space 44 can take various forms and may be made of one or more layers of flexible or rigid materials.

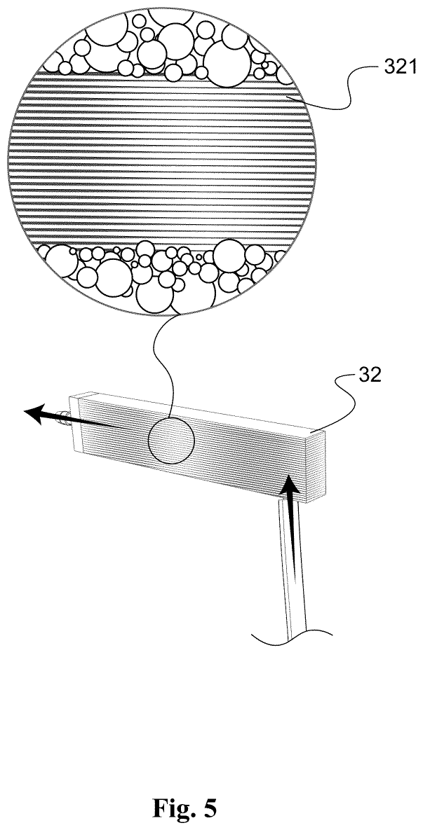

The ozone generating module 2 is configured to remain within the housing 4 at all times and to communicate with other components. The ozone recovery module 3 is one of the core functional components of the ozone disinfection device 1, directly influencing overall performance, efficiency, and environmental safety. The ozone recovery module 3 is configured to recover and filter ozone gas into harmless oxygen or gas containing a lower concentration of ozone. The ozone recovery module 3 includes a suction device 31 and a filter 32. The suction device 31 may take various forms; in this embodiment, the suction device 31 is configured to be one or more fans or pumps. The filter 32 includes a filter material 321, and the filter material 321 is configured to adsorb and decompose ozone gas (as shown in FIG. 5) and may be implemented in various ways. For example, in certain embodiments, the filter 32 of the ozone disinfection device 1 employs a combination of multiple filter materials 321 to achieve better filtration. These multiple filter materials 321 are arranged in layers, with each layer delivering a distinct filtering effect. By way of illustration, a physical filtration layer and a chemical adsorption layer may be provided simultaneously. The physical filtration layer is configured to screen and intercept large particulate matter, such as dust and bacteria, and may include a HEPA filter, a mesh filter layer, or a foam filter layer. The chemical filtration layer operates through chemical reactions and may include, but is not limited to, activated carbon, titanium dioxide, manganese compounds, or silica gel adsorption layers. Through the coordinated use of multiple filter materials 321, the filter 32 enables a more comprehensive air purification effect. In this manner, the filter 32 not only removes large particulate matter but also adsorbs smaller gases and fine particles, thereby enhancing the purification capability and overall performance of the ozone disinfection device 1.

Additionally, multiple layers of the same filter material 321 may be used to repeatedly filter a single substance, thereby achieving a more thorough filtration effect. In other embodiments, the filter 32 is formed with only a single layer of filter material 321; in this configuration, the filter material 321 is required to provide a robust filtration effect.

The filter material 321 may be selected from high-efficiency catalysts (such as manganese dioxide, platinum, or rhodium), common catalysts (such as activated carbon or alumina), or a combination thereof. Moreover, certain molecular sieve materials effectively adsorb ozone, thereby serving a purification function in the ozone disinfection device 1. In other forms, the filter 32 includes a HEPA filter material to adsorb fine particles, ensuring unimpeded airflow while preventing the spread of harmful substances.

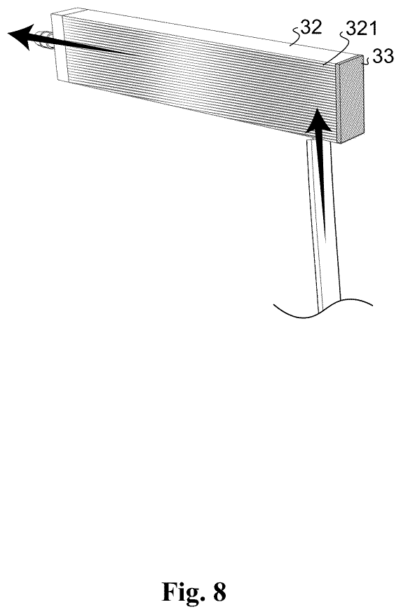

Generally, materials used for filtration in the filter 32 have one or more of the following characteristics: high porosity, providing a large surface area to facilitate ozone adsorption and catalytic decomposition; high stability, ensuring durability during ozone decomposition with strong thermal and chemical resistance; high catalytic activity, achieved through surface treatment or modification to enhance the decomposition of ozone into oxygen; and environmental friendliness, ensuring the material is non-toxic and safe for effective ozone filtration. Both sides of the filter 3 may include cotton or mesh to secure the filter material 321, improving the stability and reliability of the filter, as shown in FIG. 8. Additionally, a unique structural design may be implemented to hold the filter material 321 in place.

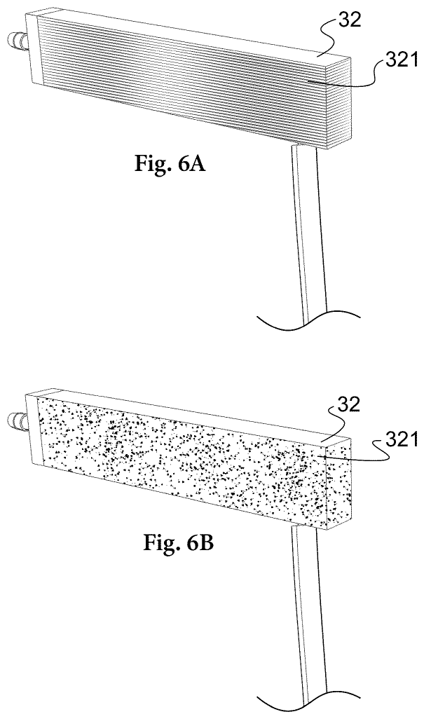

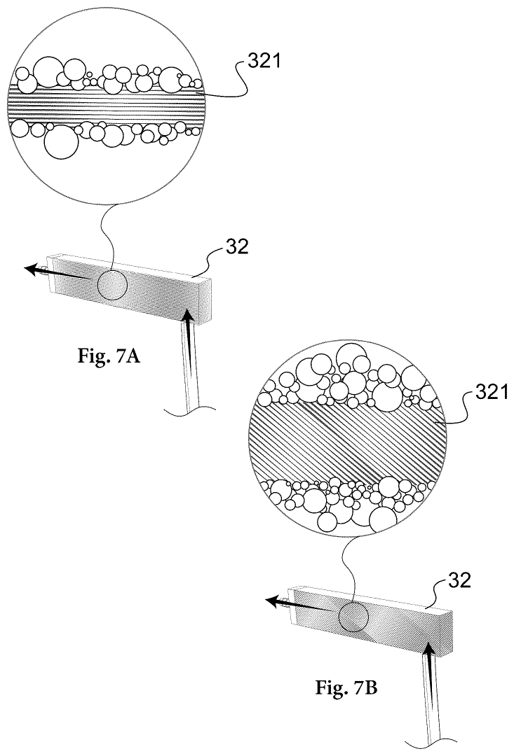

To ensure the effectiveness of the filter material 321, certain properties of the filter material 321 in the filter 32 are specified. For example, in this embodiment, the filtration efficiency of the ozone recovery module 3 is specified to be positively correlated with the total surface area of the filter material 321. The term “filtration efficiency” refers to the percentage of the target substance removed by the filter material. It can be defined as: Filtration Efficiency (%)=[(Mass of ozone gas entering the filter-Mass of ozone gas after filtration)/Mass of ozone gas entering the filter]×100%. The filter material 321 in the filter 32 has a filtration efficiency of at least 70%. Its specific surface area ranges from 5 m²/g to 500 m²/g, and preferably from 50 m²/g to 200 m²/g. Specific surface area refers to the total surface area per unit mass of a material, generally expressed in m²/g or cm²/g. It includes both external surface area (the outer surface of the filter material 321) and the surface area of internal pores (found in porous structures). A filter material 321 with a higher specific surface area provides a larger surface for capturing and adsorbing ozone gas, thereby ensuring a higher level of cleanliness. Conversely, a filter material 321 with a moderate specific surface area delivers effective filtration without creating excessive flow resistance, allowing air to pass smoothly through the ozone filter material 321. Through experimental verification, the present disclosure controls the specific surface area of the filter material 321 within an effective range to enhance the reliability of the ozone disinfection device 1 (see FIGS. 6A, 6B, 7A and 7B, where FIGS. 7A and 7B illustrate how different filter materials 321 in the ozone filter 3 adsorb ozone gas).

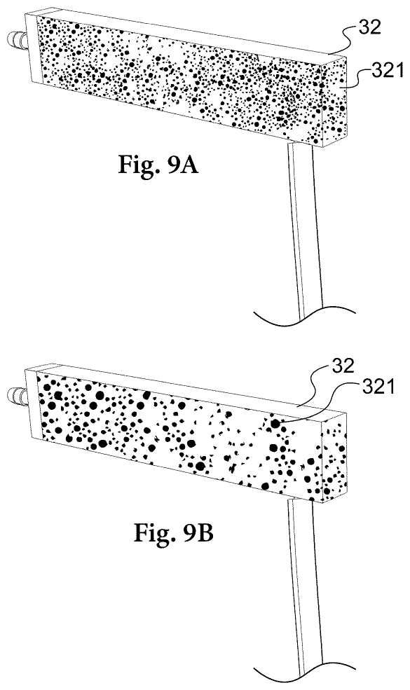

In certain embodiments, the filter material in the filter 3 is manganese dioxide, having a specific surface area in a range of 50 m²/g to 300 m²/g, or alumina, having a specific surface area in a range of 200 m²/g to 300 m²/g. The porosity of the filter 32 ranges from 10% to 95%, preferably from 50% to 85%. Porosity here refers to the ratio of internal void volume of the filter material 321 to its total volume, typically expressed as a percentage. A higher porosity indicates more voids in the material, providing greater airflow space and reducing flow resistance, but potentially compromising the structural stability of the filter material 321 and reducing its durability, which may in turn lower filtration effectiveness. Conversely, a lower porosity is configured to more effectively adsorb or capture additional ozone gas; however, it may cause excessively narrow filtration channels, restricting airflow and reducing the filtration efficiency of the ozone recovery module 3. If the porosity is too low, the filter material 321 may become clogged, decreasing its long-term reliability (see FIGS. 9A and 9B, illustrating filter material 321 with different porosity levels in the ozone filter 3). These porosity data have also been tested to determine an optimal porosity level for the filter material 321. Furthermore, the weight of the filter material 321 should be greater than 1.55 g to ensure its filtration quality.

The ozone recovery module 3 further includes at least one exhaust port 41 configured to discharge gas. The at least one exhaust port 41 is located downstream of the ozone disinfection space 44, and specifically, downstream of the filter 32. The at least one exhaust port 41 is configured to connect to the ozone recovery module 3 to ensure that the discharged ozone has been treated and converted into harmless oxygen or low-concentration ozone, thereby meeting environmental protection requirements.

The ozone generating module 2, suction device 31, and filter 32 are intercommunicable. These three components may be connected through an ozone distribution pipeline 45. The ozone distribution pipeline 45 is configured as any conduit form that allows gas flow, enabling the components to connect and facilitating the transmission of gas between them. The ozone distribution pipeline 45 is made from corrosion-resistant and impermeable materials to prevent ozone leakage during transmission, thereby protecting the device and ensuring user safety. The ozone distribution pipeline 45 is arranged flexibly to align with the layout of functional components within the independent housing 4, enabling optimal gas transport paths in various spatial configurations. In some embodiments, the ozone distribution pipeline 45 may also be used in conjunction with check valves or control valves. These valves are configured to regulate the flow rate of the gas and prevent backflow of ozone gas, further enhancing the safety and reliability of the ozone disinfection device 1.

Under normal circumstances, the ozone generating module 2 and the ozone recovery module 3 are located within the same space of the ozone disinfection device 1. The relative positions of the ozone generating module 2, the suction device 31, and the filter 32 can be arranged in various configurations. Among these configurations, the suction device 31 and the filter 32 are positioned downstream of the ozone generating module 2. The term “downstream” is defined based on the flow path of the gas and can be understood as any position where the suction device 31 and the filter 32 are located after the ozone gas flows out of the ozone generating module 2. In one configuration, the suction device 31 is positioned downstream of the filter 32. In this arrangement, ozone gas is first filtered by the filter 32, which helps prevent high concentrations of ozone gas or particulate matter from entering the suction device 31. This reduces contamination of the suction device 31, thereby extending its operational lifespan. In another configuration, the suction device 31 may also be positioned upstream of the filter 32.

In addition to the components described above, a control system is typically provided inside the housing 4 of the ozone disinfection device 1. The control system is configured to coordinate and manage stable operation of each of the aforementioned components, and to automatically adjust the operating states of the ozone generating module 2, the suction device 31, the filter 32, and other parts according to preset modes. This arrangement ensures that the device performs the disinfection process efficiently and safely.

Furthermore, in some instances, the control system is configured to implement a fault diagnosis function, enabling it to detect anomalies promptly and trigger alarms or initiate shutdown protection to prevent device damage or harm to the environment and users.

When the ozone disinfection device 1 includes an alarm function, this function typically cooperates with a user interface. Specifically, if a malfunction occurs, the alarm system is configured to interact with other components inside the housing 4 to locate and pre-treat the problematic area swiftly. This approach prevents further damage caused by extended operation and facilitates subsequent repairs by service personnel. The alarm system thus enhances user safety, optimizes the user experience, and ensures more secure and reliable disinfection. In addition, the alarm system is configured to work in conjunction with the user interface to provide users with timely notifications and reminders.

The ozone disinfection device 1 may further include an ozone operation system that provides users with a more convenient operation experience and enhanced functionality feedback. The operation system includes, but is not limited to, one or more display screens, indicator lights, buttons, and interfaces. These components offer simplified operation and more comprehensive functional indications for the user.

The display screens, indicator lights, and other user interfaces are configured to display the operating progress of the ozone disinfection device 1, such as the percentage of total operating time completed or a countdown of remaining operating time. They may also be used to indicate parameter settings and fault warnings, enabling users to clearly understand the current operating progress and status of the device. The indicator lights, in particular, may be designed to alert users to the device's status through different colors or flashing patterns. In other embodiments, the ozone disinfection device 1 may not include a related ozone operation system for control. Instead, it may feature alternative control methods, such as a voice control system.

During the operating period of the ozone disinfection device 1 in the present disclosure, the volume of ozone gas generated by the ozone generating module 2 is sufficient to maintain an ozone concentration in an external disinfection space 44 that is not lower than the threshold required to kill typical germs inside a respiratory device and its accessories, thereby ensuring complete elimination of such germs. These accessories include, but are not limited to, a mask, a nasal mask, a hose, or a frame used in conjunction with a respirator. Further, throughout the operating time of the ozone disinfection device 1, the volume of gas drawn in by a suction device 31 is greater than or equal to the volume of ozone gas generated by an ozone generating module 2 during the same period. This arrangement ensures that the ozone gas in the external disinfection space 44 is fully converted into a harmless gas by the filter 32, preventing harm to humans or the environment.

In this embodiment, the disinfection path of the ozone disinfection device 1 is as follows: an ozone generating module 2 inside a housing 4 produces ozone gas, which is delivered through an ozone distribution pipeline 45 to an interface 42. The ozone gas is then transferred through a hose connected to the interface 42 to the external disinfection space 44 for disinfecting a respiratory device and its accessories. After disinfection, the gas is recovered through one or more suction devices 31 through the same hose into an ozone recovery module 3 located inside the housing 4, and following reaction, the gas is discharged through a channel in the housing 4. The hose may be any form of tubing configured to convey gas.

In other embodiments, during the operating period of the ozone disinfection device 1, both the suction device 31 and the filter 32 are in the same operational or non-operational state. This can be understood as the suction device 31 and the filter 32 being configured to operate simultaneously.

In other embodiments, during the ozone recovery process, the hose used to recover ozone gas is not the same as the hose used to deliver ozone to the external disinfection space 44.

In other embodiments, the ozone disinfection device 1 may include more than one filter 32. Multiple filters 32 can be positioned in different locations within the ozone disinfection device 1. For example, in one scenario, the ozone disinfection device 1 includes at least two filters 32, each configured to connect separately to the external disinfection space 44 and a hose. These two filters 32 may have the same or different functional effects.

In other embodiments, the filter 32 incorporates self-cleaning technology, such as removing particles attached to the filter 32 through reverse airflows or vibrations, thereby extending the lifespan of the filter 32.

### Embodiment 2

The ozone disinfection device 1 in this embodiment includes an ozone generating module 2 configured to generate and output ozone gas, and an ozone recovery module 3 located downstream of the ozone generating module 2. The ozone recovery module 3 includes a suction device 31 and a filter 32 and is configured to draw in and filter ozone gas. The device also includes a control system configured to control the operation of its various components. The control system includes at least one sensor.

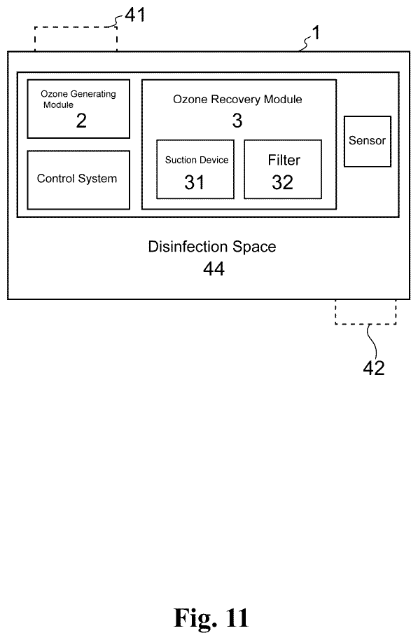

This embodiment differs from Embodiment 1 in that it includes at least one sensor to monitor ozone gas (as shown in FIG. 11). In FIG. 11, the sensor illustrated is an ozone concentration sensor. The ozone concentration sensor is configured to monitor the concentration of ozone gas at various locations within the ozone disinfection device 1 in real time. This ensures that the ozone concentration produced by the ozone generating module 2 remains within a normal range, preventing potential health or ecological hazards caused by anomalies in the ozone control system. The ozone concentration sensor can interact with other components within the housing 4, such as the ozone generating module 221, to achieve an intelligent ozone concentration output that can be adjusted by the user, providing greater operational flexibility. This interaction also reduces energy or material waste, improves the safety and reliability of the device, and enhances user confidence in the product. The sensor in the ozone disinfection device 1 can also work in conjunction with an alarm system. The alarm function is configured to notify the user visually or audibly in the event of abnormal monitoring by the sensor or improper usage by the user, thereby preventing potential greater hazards. Abnormal conditions may include excessively high ozone concentration, overheating of components, failure or clogging of the filter 32, electrical current anomalies, component malfunctions, or sealing leaks.

The sensor can be located at any position in the ozone disinfection device 1 to perform various functions. For example, it may be positioned within the disinfection space to ensure that the ozone concentration during disinfection remains within an effective and safe range, preventing harm to the environment from excessive ozone concentrations or suboptimal disinfection caused by insufficient ozone levels. The sensor can also be located at the gas recovery point to monitor the quality of the recovered air and ensure that residual ozone has been fully removed.

In other embodiments, the ozone control system may also have other types of sensors. These include, but are not limited to, include, one or more of the following: a temperature and humidity sensor, a pressure sensor, an oxygen sensor, a flow sensor, a VOC sensor, a particulate sensor, a current sensor, or a safety lock sensor.

### Embodiment 3

This embodiment of the ozone disinfection device 1 includes an ozone generating module 2, configured to produce and output ozone gas, and an ozone recovery module 3, located downstream of the ozone generating module 2. The ozone recovery module 3 includes a suction device 31 and a filter 32, and is configured to draw in and filter ozone gas. The device also includes a control system, which controls the operation of various components of the ozone disinfection device 1. The filter 32 of the ozone disinfection device 1 is configured to be replaceable.

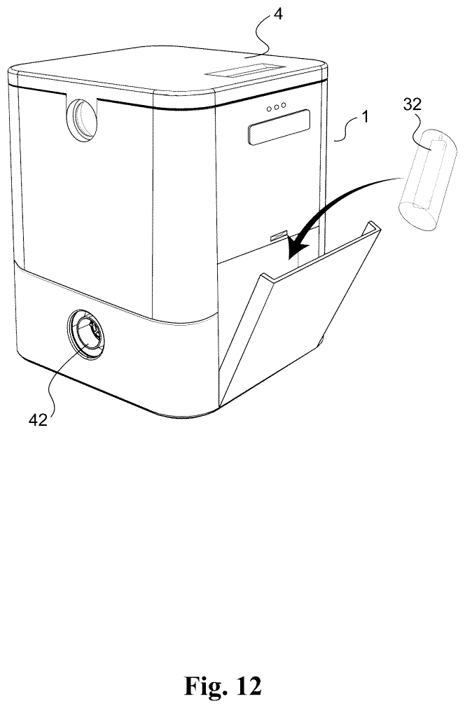

This embodiment differs from Embodiment 1 in that the filter 32 is replaceable and has a modular design that makes maintenance easier for users (see FIG. 12). The service life of the filter 32 primarily depends on the ozone concentration, usage frequency, and operating conditions. Generally, as the ozone disinfection device 1 is used over time, the filter 32 gradually adsorbs ozone gas, causing its filtration efficiency to decline. To maintain the effectiveness and safety of the ozone disinfection device 1, the housing 4 and the filter 32 are designed to fit together in a way that allows convenient replacement of the filter 32. In one scenario, the housing 4 has an openable and closable panel at the position where the filter 32 is installed, enabling a user to easily open the panel to remove and replace the filter 32. The filter 32 itself adopts a detachable, assembled structure, making the replacement process quick and straightforward. Through this modular structure, users can periodically check the status of the filter 32 on their own and determine whether it remains usable. Users can also promptly replace the filter 32 upon failure or the end of its service life, without having to dismantle or discard the entire device. This not only increases maintenance efficiency but also reduces repair costs. Furthermore, the replaceable design of the filter 32 enhances the device's sustainability, allowing the ozone disinfection device 1 to maintain a high level of disinfection efficiency and environmental performance over prolonged use. Users can keep the device in optimal working condition through simple operations, ensuring long-term stability and reliability.

In other embodiments, the ozone recovery module 3 or the filter 32 is configured as an independent part that is not combined with other functional components inside the housing 4. For example, if the disinfection space 44 of the ozone disinfection device 1 is external—meaning this disinfection space 44 is separate from the other components of the ozone disinfection device 1—the ozone recovery module 3 or the filter 32 can be provided as a standalone component configured to connect to the disinfection space 44. In this situation, the ozone generating module 2 and the ozone recovery module 3 are located within different housings of the ozone disinfection device 1.

Moreover, it is possible to combine the technical features from each of the above embodiments as needed to obtain an ozone disinfection device 1 that includes all or some of these features.

Implementing the ozone disinfection device 1 of the present disclosure has at least the following beneficial effects:
1. Compared to existing ozone disinfection devices on the market, the ozone disinfection device of the present disclosure employs more efficient filtration technology, delivering an enhanced user experience and achieving more thorough removal of ozone. As a result, the discharged gas is more environmentally friendly. Specifically, the filter of this disclosure uses optimized materials and structural designs, which may include multiple combined layers to realize different filtration functions (such as a physical filtration layer and a chemical adsorption layer). In addition, its structure is arranged to effectively improve ozone decomposition and filtration efficiency. Furthermore, the optimized filter design in the present disclosure reduces the need for frequent replacement of either the device or the filter, maximizing the operational effectiveness of each filtration layer. This lowers user costs and material waste, thereby decreasing maintenance expenses and making the device more eco-friendly. Through this design, the overall performance of the ozone disinfection device is improved while aligning with environmental protection and sustainable development requirements. Not only does it achieve a functional breakthrough, but it also contributes positively to environmental conservation, resource saving, and sustainability, making the device a more efficient, safer, and eco-friendly disinfection solution.
2. In the present disclosure, various parameters of the filter material are specified to achieve optimal filtration performance. First, the specific surface area of the filter material is controlled within a range of 5 m²/g to 500 m²/g. and preferably within a range of 50 m²/g to 200 m²/g. This not only achieves a better balance between the performance and cost of the ozone recovery module but also enhances the adaptability and application range of the filter material. A filter material with a higher specific surface area provides a larger surface for capturing and adsorbing ozone gas, thereby ensuring a higher level of cleanliness. Conversely, a filter material with a moderate specific surface area delivers effective filtration without creating excessive flow resistance, allowing air to pass smoothly through the ozone filter material. Through extensive experimental validation, preferably, the effective range for the specific surface area of the filter material is determined to be in a range of 5 m²/g to 500 m²/g. The optimization of the specific surface area achieves improved performance in terms of filtration efficiency, multifunctionality, airflow resistance, cost optimization, service life, and environmental sustainability. Furthermore, the porosity of the filter is specified to range from 10% to 95%, preferably from 50% to 85%. A higher porosity indicates more voids in the material, providing greater airflow space and reducing flow resistance. However, this may compromise the structural stability of the filter material, reducing its durability and potentially lowering filtration effectiveness. Conversely, a lower porosity is configured to more effectively adsorb or capture additional ozone gas. However, excessively low porosity may result in overly narrow filtration channels, restricting airflow and reducing the filtration efficiency of the ozone recovery module. If the porosity is too low, the filter material may also become clogged, decreasing its long-term reliability. Through extensive experimentation, it has been determined that the specified range of porosity achieves an optimal balance between filtration performance, reliability, and other critical factors. Additionally, it is established that, during the operating period of the ozone disinfection device, the volume of gas drawn in by the suction device must be greater than or equal to the volume of the ozone gas generated by the ozone generating module during the same period. This ensures the effective operation of the suction device during use, preventing the release of ozone gas at excessively high concentrations into the external environment, which could pose harm to both the environment and the user.
3. The present disclosure integrates sensors into the ozone disinfection device, enhancing its intelligence, automation, and safety. This optimization improves the disinfection effect while ensuring safety and environmental protection during use. The sensors include, but are not limited to, one or more of the following: an ozone concentration sensor, a temperature and humidity sensor, a pressure sensor, an oxygen sensor, a flow sensor, a volatile organic compound (VOC) sensor, a particulate sensor, a current sensor, or a safety lock sensor. The integration of these sensors not only enhances the multifunctionality of the device but also provides users with a more intelligent and efficient operational experience. Specifically, the ozone concentration sensor is configured to monitor the ozone concentration within the device in real time, ensuring that the ozone concentration during disinfection or recovery remains at appropriate levels. This prevents insufficient disinfection due to low ozone concentrations or harm to humans or the environment caused by excessively high concentrations. Furthermore, the ozone concentration sensor can detect whether the ozone disinfection device is leaking, thereby ensuring the effectiveness of the device's disinfection process. The temperature and humidity sensor ensures that the conditions for ozone disinfection are suitable and optimizes the disinfection environment, thereby improving the efficiency of ozone disinfection. The pressure sensor and flow sensor enable precise control of airflow distribution within the disinfection space, ensuring that ozone gas effectively reaches every part of the components being disinfected for thorough disinfection. The oxygen sensor detects the oxygen content in the recovered ozone disinfection space, helping to verify whether the ozone has been fully recovered, which benefits environmental protection and green design. Additionally, the oxygen sensor can monitor and adjust the efficiency of ozone generation, thereby optimizing the effectiveness of ozone disinfection. Other sensors, such as VOC sensors and particulate sensors, further enhance the device's ability to monitor its internal environment and make real-time adjustments to address potential contamination. The integration of current sensors and safety lock sensors further improves the safety of the device, preventing misuse and ozone gas leakage. The inclusion of one or more of these sensors enhances the intelligence and reliability of the device, providing users with a safer, more reliable, and more effective ozone disinfection solution.
4. The filter in the ozone disinfection device of the present disclosure is configured to be replaceable, offering multiple advantages such as facilitating the maintenance and management of the ozone disinfection device, as well as enhancing its disinfection efficiency and service life. First, the replaceable filter increases the variety of filters that can be used with the ozone disinfection device. Users can match filters with different internal filtering materials based on various usage scenarios and specific needs. For example, standard ozone filters can be used by general users in everyday environments. Conversely, users who frequently use ozone disinfection devices or disinfect heavily polluted devices may select filters with more efficient filtering materials. Additionally, users can choose filters with enhanced specific functions depending on the environment. For instance, in humid conditions, filters with enhanced drying functionality can be used. This approach reduces the impact of environmental factors on the filter in the ozone disinfection device, thereby improving the device's service life and disinfection efficiency to a certain extent. The ozone filter, as one of the most important functional components of the ozone disinfection device, offers additional benefits as technology advances. Users can upgrade the ozone disinfection device by replacing the filter with a more efficient or multifunctional one, providing advantages for both users and manufacturers. This approach not only reduces costs but also enhances the adaptability and versatility of the device. Furthermore, users can replace the filter independently based on their needs without requiring professional assistance, making the maintenance of the ozone disinfection device simpler and more convenient. In one approach, users can visually monitor the status of the filter and easily determine when replacement is needed, providing a more intuitive and user-friendly experience. Regular and straightforward replacement of the filter by the user prevents performance degradation caused by blockages or aging, effectively protecting and extending the service life of the entire ozone disinfection device. Finally, this method represents an environmentally friendly design for ozone disinfection devices. Instead of replacing the entire ozone disinfection device, only the smaller component—the ozone filter—needs to be replaced. This reduces the number of discarded devices and allows the ozone disinfection device to be reused multiple times with new filters. This approach aligns with modern green design principles and is more environmentally friendly.

The above description of the embodiments of the disclosure is provided with reference to the accompanying drawings. However, the disclosure is not limited to the specific embodiments described above. These specific embodiments are merely illustrative and not restrictive. Those skilled in the art, in light of the teachings of the disclosure, may make many modifications and variations without departing from the spirit and scope of the disclosure as defined by the claims. All such modifications and variations are within the protection scope of the disclosure.

It must be noted that as used herein and in the appended claims, the singular forms “a”, “an”, and “the” include their plural equivalents, unless the context clearly dictates otherwise.

1. An ozone disinfection device with an ozone recovery module, configured to be connectable to a hose or a respiratory device to disinfect the hose or the respiratory device, the ozone disinfection device comprising:
an ozone generating module, configured to generate and output ozone gas;
the ozone recovery module, located downstream of the ozone generating module, wherein the ozone recovery module comprises a suction device and a filter, and wherein the ozone recovery module is configured to draw in and filter the ozone gas;
a control system, configured to control operation of components of the ozone disinfection device;
wherein the ozone generating module, the suction device, and the filter are configured to be in communication with one another, and
wherein the filter comprises a filter material, and a filtration efficiency of the ozone recovery module is positively correlated with a total surface area of the filter material.

2. The ozone disinfection device according to claim 1, wherein the control system further comprises at least one sensor.

3. The ozone disinfection device according to claim 1, wherein the ozone recovery module further comprises at least one exhaust port, the at least one exhaust port being configured to discharge gas.

4. The ozone disinfection device according to claim 3, wherein the at least one exhaust port is located downstream of the filter.

5. The ozone disinfection device according to claim 1, wherein, during an operating period of the ozone disinfection device, a volume of the gas drawn in by the suction device is greater than or equal to a volume of the ozone gas generated by the ozone generating module during the same period.

6. The ozone disinfection device according to claim 1, wherein the filter is configured to be replaceable.

7. An ozone disinfection device with an ozone recovery module, configured to be connectable to a hose or a respiratory device to disinfect the hose or the respiratory device, the ozone disinfection device comprising:
an ozone generating module, configured to generate and output ozone gas;
the ozone recovery module, located downstream of the ozone generating module, wherein the ozone recovery module comprises a suction device and a filter, and wherein the ozone recovery module is configured to draw in and filter the ozone gas;
a control system, configured to control operation of components of the ozone disinfection device; and
wherein the filter comprises a filter material, and the filter material has a specific surface area in a range of 5 m²/g to 500 m²/g.

8. The ozone disinfection device according to claim 7, wherein the ozone generating module, the suction device, and the filter are configured to be in communication with one another.

9. The ozone disinfection device according to claim 7, wherein the control system comprises at least one sensor.

10. The ozone disinfection device according to claim 9, wherein the at least one sensor is configured to detect ozone concentration.

11. The ozone disinfection device according to claim 7, wherein the filter is located downstream of the ozone generating module.

12. The ozone disinfection device according to claim 7, wherein, during an operating period of the ozone disinfection device, the suction device and the filter are configured to operate and cease operation simultaneously.

13. An ozone disinfection device with an ozone recovery module, configured to be connectable to a hose or a respiratory device to disinfect the hose or the respiratory device, the ozone disinfection device comprising:
an ozone generating module, configured to generate and output ozone gas;
the ozone recovery module, located downstream of the ozone generating module, wherein the ozone recovery module comprises a suction device and a filter, and wherein the ozone recovery module is configured to draw in and filter the ozone gas;
a control system, configured to control operation of components of the ozone disinfection device; and
wherein the filter is configured to have a porosity in a range of 10% to 95%.

14. The ozone disinfection device according to claim 13, wherein the ozone generating module, the suction device, and the filter are configured to be in communication with one another.

15. The ozone disinfection device according to claim 13, wherein the control system comprises at least one sensor.

16. The ozone disinfection device according to claim 15, wherein the ozone generating module and the ozone recovery module are arranged in different compartments within the ozone disinfection device.

17. The ozone disinfection device according to claim 13, wherein the ozone generating module and the ozone recovery module are arranged in a same compartment within the ozone disinfection device.

18. An ozone disinfection device with an ozone recovery module, configured to be connectable to a hose or a respiratory device to disinfect the hose or the respiratory device, the ozone disinfection device comprising:
an ozone generating module, configured to generate and output ozone gas;
the ozone recovery module, located downstream of the ozone generating module, wherein the ozone recovery module comprises a suction device and a filter, and wherein the ozone recovery module is configured to draw in and filter the ozone gas;
a control system, configured to control operation of components of the ozone disinfection device;
wherein the ozone generating module, the suction device, and the filter are configured to be in communication with one another, and
wherein the control system comprises at least one sensor.

19. The ozone disinfection device according to claim 18, wherein the ozone recovery module further comprises at least one exhaust port, the at least one exhaust port being configured to discharge gas.

20. The ozone disinfection device according to claim 18, wherein the filter comprises a filter material, and the filter material has a filtration efficiency of at least 70%.

21. The ozone disinfection device according to claim 20, wherein the filter material has a porosity in a range of 50% to 85%.

22. The ozone disinfection device according to claim 18, wherein the at least one sensor comprises one or more of an ozone concentration sensor, a temperature and humidity sensor, a pressure sensor, an oxygen sensor, a flow sensor, a volatile organic compound sensor, a particulate sensor, a current sensor, or a safety lock sensor.
